(19) United States
(12) Patent Application Publication
(10) Pub. No.: US 2021/0267782 A1
(43) Pub. Date: Sep. 2, 2021

(54) FINGER ORTHOSIS AND METHOD FOR PRODUCTION THEREOF

(71) Applicant: Ottobock SE & Co. KGaA, Raubling (DE)

(72) Inventor: Christian KIENZLE, Raubling (DE)

(73) Assignee: Ottobock SE & Co. KGaA, Duderstadt (DE)

(21) Appl. No.: 17/052,777

(22) PCT Filed: May 3, 2019
(86) PCT No.: PCT/EP2019/061406

§ 371 Date: Nov. 3, 2020

(30) Foreign Application Priority Data
May 4, 2018 (DE) 10 2018 206 904.2

(51) Int. Cl. A61F 5/01; A61F 5/058; B33Y 80/00

(52) U.S. Cl. CPC A61F 5/0118; B33Y 80/00; A61F 5/05875

(57) ABSTRACT

A finger orthosis for applying a force to a structural part using a finger. Finger orthoses of this kind can be used as work aids or assembly aids. A method for producing such finger orthoses is also disclosed. The finger orthosis has a support element for supporting the finger orthosis on at least one further finger. In a further variant, the finger orthosis has a tool, connected to the frame, for transmitting the force to a structural part.

The invention relates to a finger orthosis for exerting a force on a component by means of a finger. Such finger orthoses can in particular be used as work aids or as assembly aids. The invention furthermore also relates to a method of manufacturing such finger orthoses.

Certain work steps or assembly steps can be carried out by an operator, in particular with a finger. In so doing, the operator exerts a force with his finger on a component, for example, via a tool. If the work steps or assembly steps are carried out repeatedly, stress symptoms of the finger tissue, the ligaments, the muscles, etc. can occur.

It is therefore the object of the present invention to provide a finger orthosis and a method of manufacturing a finger orthosis as a work aid or assembly aid that makes it possible to repeatedly exert a force on a component by a finger without causing stress symptoms at the finger.

This object is achieved by a finger orthosis in accordance with claim 1 or claim 4 and by the method in accordance with claim 17. Advantageous further developments of the finger orthosis in accordance with the invention and of the method in accordance with the invention are provided in the dependent claims.

The finger orthosis in accordance with the invention for exerting a force on a component by means of a finger comprises a frame for receiving a finger in an embodiment such that the frame at least partially circularly surrounds a fingertip and a side of a distal interphalangeal joint remote from the fingertip substantially perpendicular to a longitudinal direction of the finger. The frame thus forms a cuff for holding the frame at the fingertip and at the distal interphalangeal joint. The frame furthermore at least partially circularly surrounds the finger from the fingertip to the side of the finger end joint remote from the fingertip substantially in a plane of the longitudinal direction. The two cuffs are thus connected to one another and the frame is thus formed in a contiguous manner.

A finger is understood here both as the four different fingers and the thumb of a hand. To the extent that the invention specifically relates to a finger orthosis for a thumb, the fingertip is in particular understood as a thump tip, the distal interphalangeal joint as a distal interphalangeal joint of the thumb, and both a proximal interphalangeal joint and a metacarpophalangeal joint as a metacarpophalangeal joint of the thumb.

In a first variant of the finger orthosis in accordance with the invention, the finger orthosis now has a tool that is connected or connectable in one piece or in multiple parts to a frame for transferring the force onto the component.

In a second variant of the finger orthosis in accordance with the invention that can be combined without restriction with the first variant the finger orthosis has at least one support element for supporting the orthosis at least one further finger, advantageously a clip or a protuberance. If the support element is a clip, it is advantageously configured such that it engages around the finger that is held by the finger orthosis at its front side and is connected to the frame at least one side. A plurality of support elements can also be provided.

This support element serves as a support for at least one further finger, in particular an adjacent finger, possibly not embraced by the orthosis, that thus supports the finger embraced by the orthosis. The forces on the orthosis are thus not only taken up by the surrounded finger, but are rather led into the entire hand by the supporting, adjacent finger.

The support element can in particular be configured as a cushion or can include a cushion to distribute the forces acting on the orthosis over a greater area.

The support element can furthermore be designed as slip-resistant, for example by selection of a suitable slip-resistant material, a coating with a suitable slip-resistant material, or a surface treatment of the support element for increasing the grip of the surface of the support element.

The support element can be designed such that one or a plurality of fingers of the same hand as the finger received in the orthosis, optionally by a frame of the orthosis, or one, more, or all the fingers of the other hand can be applied to the support element or can be received by it. The support element can in particular be configured such that the finger to be applied or some or all of the fingers to be applied can be loosely applied to the orthosis, i.e. without a fixed connection (i.e. without a shape matched connection with the exception of the application direction of the respective finger at the support element, a connection with material continuity, or the like). This finger or these fingers thus differ from that finger for whose reception, optionally by a frame of the orthosis, the orthosis is provided and configured.

The finger orthosis in accordance with the invention therefore makes it possible that, during the performance of manual work steps or assembly steps with a finger, the force acting on a region of the finger is distributed over the whole finger or over the hand and stress on the region of the finger, for example of a finger tissue, of ligaments, of muscles, etc. of the finger is avoided. The finger orthosis in accordance with the invention furthermore makes possible a direct and targeted force exertion on a component without the aid of additional tools.

The finger orthosis in accordance with the invention itself and/or the support element can be a component of a glove or can be integrated in a glove in every possible embodiment.

In a preferred embodiment of the invention, the frame can be produced in one piece. This makes possible a particularly simple and fast manufacture of the finger orthosis since the frame does not first have to be assembled from a plurality of parts in a laborious manner.

The frame and the tool can be connected flexibly, rigidly, and/or directly to one another and/or can be produced in one piece with one another. A rigid connection of the frame and the tool enables a direct and efficient force transmission from the finger orthosis to the component. If the frame and the tool are produced in one piece with one another, additional assembly steps can be saved in which the tool is fastened to the finger orthosis.

The frame can be formed over the full area over the surface spanned by it and/or can have one or more passage openings. One or more passage openings can here provide a good ventilation of the finger in the finger orthosis. The passage openings can furthermore improve a fit of the finger orthosis in that they surround and are supported at these projecting regions of the finger such as a joint or bony structures.

In a further advantageous embodiment of the invention, the tool can be connected via a tool mount, in particular via a tool mount produced in one piece with the frame. It can here in particular be a universal tool mount that enables a fastening of different tools.

It is particularly advantageous if the frame is not fully circularly closed in a region surrounding the distal interphalangeal joint. This enables a flexible adaptation to different finger circumferences and thus a firm seat of the finger orthosis for different finger sizes.

The frame can furthermore comprise or consist of a flexible material, a metal, and/or a plastic, in particular polyamide, polyamide 11, polyamide 12, and/or thermoplastic polyurethane (TPU). The tool mount can comprise or consist of a metal, in particular stainless steel.

In a further advantageous embodiment of the invention, a wall can be arranged within the frame that is firmly connected to the frame and that extends over a surface of the finger orthosis spanned by the frame. The wall can furthermore also extend beyond the surface spanned by the frame.

The wall can furthermore be closed over the full area over the surface spanned by the frame and/or can have one or more passage openings, with the passage openings in particular corresponding to the passage openings of the frame. In this case, the common passage openings through the frame and the wall can serve the ventilation of the finger.

The wall can in particular include or consist of a flexible material and/or a plastic, in particular thermoplastic polyurethane (TPU), with the wall in particular being able to have a wall thickness of 0.5 to 2 mm, in particular of 1 mm.

A further embodiment of the finger orthosis in accordance with the invention provides that the frame is configured to take up in itself a region of the finger that extends from a distal interphalangeal member up to a wrist, with the frame in particular being able to have an orthotic joint in a region between a proximal interphalangeal joint and the wrist. Such a design of the finger orthosis makes it possible to distribute the force exerted on the component via a finger orthosis by the operator over a hand and to thereby be able to exert greater forces on the component without stressing the finger.

The tool can preferably be arranged in a region of the frame receiving the fingertip. The tool can in particular comprise a cable connector holder, a screwing aid, in particular a screwdriver, a wrench, and/or a force enhancer, in particular a pressing aid.

The present invention also includes a method of manufacturing one of the finger orthoses described above. The method in accordance with the invention comprises the following steps: Production of the frame by means of a 3D printing process, an injection molding process, and/or a laminating process, and optionally a simultaneous or later production of the tool with the frame or fastening of the tool to the frame.

A tool mount can preferably be adhesively bonded, plugged, and/or screwed to the frame for fastening a tool and the tool can be fastened to the tool mount.

The tool can also be directly adhesively bonded, plugged, or screwed to the frame, or flexibly connected to the frame.

The finger orthosis in accordance with the invention can have a plurality of further advantageous embodiments with which additional effects can be achieved.

The region of the finger orthosis that surrounds the fingertip can, for example, be produced in one part and in particular from special material such as particularly resistant material or particularly slip-resistant material, or also electrically conductive material, for example. In this manner, the tip of the finger orthosis already represents the tool in accordance with the invention integrated in the finger orthosis.

Skin contact with the tool surface, for example of the component to be assembled, can thus be avoided, for example, by a suitable design and the skin of the workman can thus be protected from its elements. Injury protection against sharp-edged components can also be implemented.

The tip of the finger orthosis can also be produced from elastic material to achieve resilience. Blows can be taken up and force peaks on the finger of the workman can thus be taken up and cushioned by such flexible elements, for example from TPU, rubber, or spiral springs, or other kinds of springs.

The tip of the orthosis can also be designed as conductive, for example by a conductive TPU inlay. Capacitive touchscreens can hereby be operated by the orthosis, for example. The required electrical circuit for inductive touchscreens can likewise be integrated in the finger orthosis. Inputs can thus also be carried out on corresponding devices without additional input devices.

Sensors or other kinds of elements for the recognition of states or a change of states can also be considered as a tool that it connected or connectable to the orthosis or to the frame of the orthosis. It is thus possible to accommodate elements in the frame of the finger orthosis that enable a recognition of the finger orthosis, in particular of its position, by others. A machine can, for example, recognize the position of a finger in this manner and can trigger or carry out or also end actions where necessary in dependence on the position of the finger.

Collisions between the user and a machine can also be recognized and avoided by such sensors, contacts, transmitters, or recognizable elements. An example for this is an orthosis with which the workman can position an element in a machine by his hand and by which a machine can recognize the position of the orthosis. The machine can therefore recognize whether the workman holds his hand in the vicinity of the machine or remote therefrom and can, where necessary, omit or stop further actions that could result in injury to the workman.

It is in particular possible by the finger orthosis in accordance with the present invention to fix the position of the finger or to restrict its mobility. The flexion angle and/or extension angle of the individual finger joints can in particular be restricted or limited. The frame can have a section for this purpose on the side of the orthosis facing the front side of the finger that extends over the finger joint to be limited such that it limits or even prevents the flexion or extension of the finger joint. The angle of flexion of the finger joints can be limited via corresponding limit stops of this section that can optionally also be designed as resilient so that only ergonomic positions can be adopted. In this respect, it can be sufficient, instead of a limitation of movement, to only provide the workman with feedback, instead of a rigid movement limitation, that he has reached the start of an unergonomic position so that the user can avoid this position and a further movement in the direction of the unergonomic position. It is consequently not necessary for this purpose that the limit stop can take up forces of any desired amount; it is rather sufficient that the limit stop provides sufficient feedback to the user.

It is furthermore possible to design the orthosis such that the workman or the user can only exert a defined force or a defined torque on the tool and thus on the component.

The required and fixed force is, for example, frequently greatly exceeded in assembly procedures; for example, a user tends to press a plug into an opening a little too firmly. Such an incorrectly applied force can cause damage, however. It is, however, also possible to attach a spring, for example, between the tool and the frame of the finger orthosis that begins to deflect and thus provides the user with feedback on how much force is actually necessary exactly from that force onward that is required for joining. This results in a training effect on the user so that he gradually becomes familiar with the correct and necessary force.

Some embodiments of a finger orthosis in accordance with the invention will be described in more detail in the following with reference to Figures. Different elements that are essential to the invention or also advantageous and further developing elements are mentioned here in each case within the framework of a specific example, with individual ones of these elements also being able to be used as such to further develop the invention—also removed from the context of the respective example and further features of the respective example.

The same or similar reference numerals are furthermore used for the same or similar elements in the Figures and their explanation has therefore been omitted in part.

There is shown in

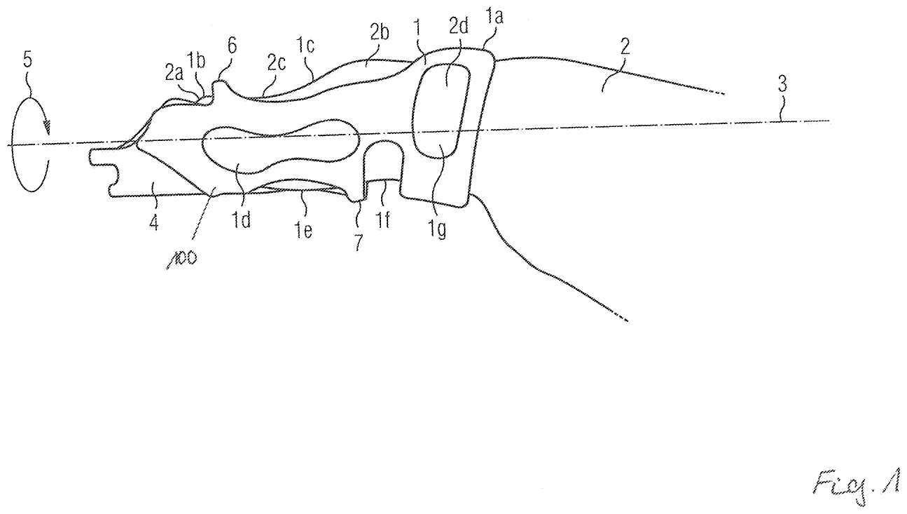

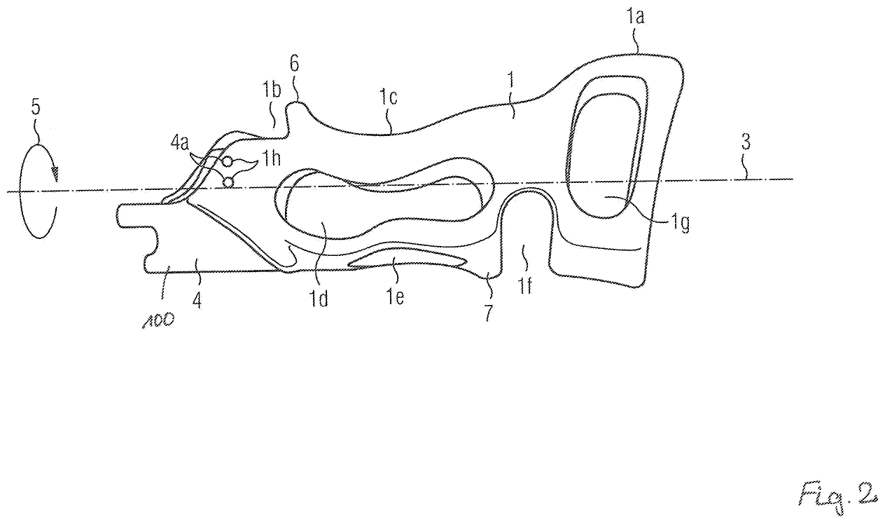

FIG. 1 a side view of a first embodiment of a finger orthosis in accordance with the invention; and

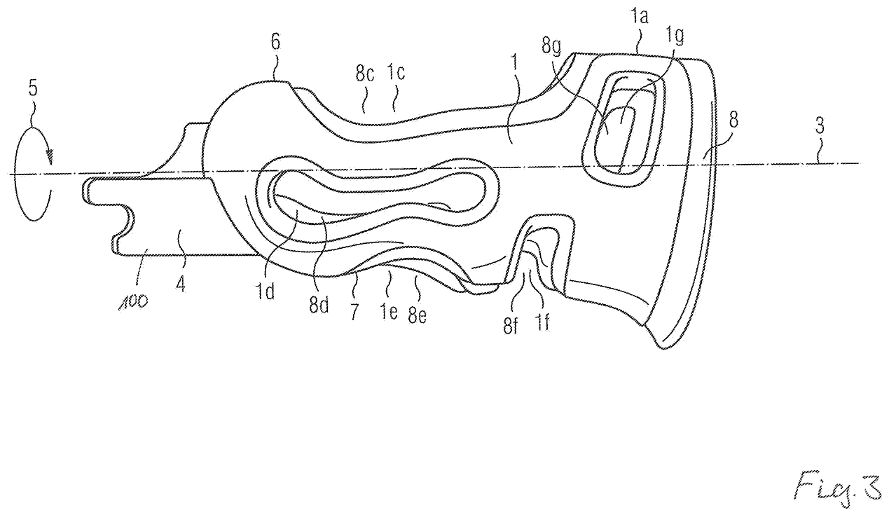

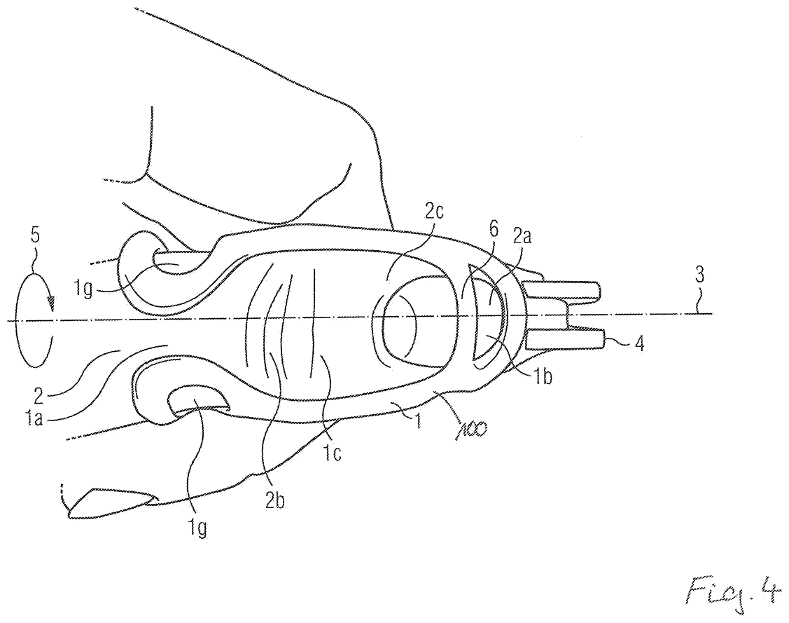

FIGS. 2 to 13 further examples of a finger orthosis in accordance with the invention respectively in an oblique plan view, side view sectional view, lower view or, plan view.

FIG. 1 shows a side view of a first embodiment of a finger orthosis 100 in accordance with the invention. The finger orthosis 100 has a stiff frame 1 for receiving a finger 2. The frame 1 substantially extends along the longitudinal direction 3 of the finger 2. The frame substantially surrounds a fingertip 2a along a plane in parallel with a palm of a hand in the region of the fingertip 2a. The frame 1 further surrounds the fingertip 2a in a peripheral direction 5 of the finger 2. At a side of a distal interphalangeal joint 2b remote from the fingertip 2a, the frame 1 largely surrounds the finger 2 except for a variable gap 1a that is designed as wider or narrower in the applied state depending on the fit of the finger orthosis 100. The frame 1 furthermore has a plurality of passage openings 1b-1g, wherein one passage opening 1b is provided in the region of the fingertip 2a on an upper side 6 of the finger orthosis 100 corresponding to an upper side of the hand, one passage opening 1c is provided on a side of the distal interphalangeal joint 2b facing the fingertip 2a on the upper side 6, one passage opening 1e is provided at a lower side 7 of the finger orthosis 100 corresponding to a lower side of the hand in the region of the distal interphalangeal joint 2c, one passage opening 1f is provided at the lower side 7 in the region of the distal interphalangeal joint 2b, and lateral passage openings 1d are provided in the region of the distal interphalangeal member 2c, and lateral passage openings 1g are provided in the region of the proximal interphalangeal joint 2d. The passage openings enable good ventilation of the finger 1, an improved fit of the finger orthosis 100 at the finger 2, and a saving of material and weight in the finger orthosis 100. In the region of the finger orthosis 100 that can receive the fingertip 2a, the finger orthosis 100 additionally comprises a tool 4. The tool 4 here comprises a cable connector holder that is adhesively bonded to the frame 1 or is laminated into the frame 1.

The finger orthosis 100 of the first embodiment shown here makes possible a plugging of a cable connector into an electrically conductive component. A compressive force of the finger has to be applied to the cable connector and to the electrically conductive component for this purpose. When repeated frequently, this work step without a finger orthosis 100 can lead to irritation of the tissue at the fingertip or to stress of the ligaments and of the muscles of the finger. The finger orthosis 100 protects the finger tissue and the ligaments and the muscles from such stress in that the stiff, supporting frame 1 distributes the compressive force acting on the fingertip 2a over the distal interphalangeal joint 2c and the proximal interphalangeal joint 2d.

FIG. 2 shows a side view of a second embodiment of a finger orthosis 100 in accordance with the invention. The finger orthosis 100 of the second embodiment only differs from the finger orthosis 100 of the first embodiment of FIG. 1 in the fastening of the tool 4 that here likewise comprises a cable connector holder. In FIG. 2, the tool is plugged into the frame 1 in the region of the fingertip and is fixed by means of pins 4a that laterally grip into small openings 1h at the side in the frame in the region of the fingertip. Openings 1h and pins 4a form a tool mount 10. Instead of the pins 4a, rivets or screws are also conceivable for fastening the tool.

FIG. 3 shows a side view of a third embodiment of a finger orthosis 100 in accordance with the invention. The finger orthosis 100 of the third embodiment differs from the finger orthosis 100 of FIG. 1 in that a wall 8, preferably of thermoplastic polyurethane (TPU), is arranged within the frame 1. The wall extends on an inner side of the frame 1 over the total area covered by the frame 1. The wall 8 likewise has a plurality of passage openings 8c-8g that correspond to the passage openings 1c-1g of the frame 1. The wall 8 furthermore slightly projects beyond the frame 1 at the margins of the frame 1. The wall 8 serves a firmer fit of the finger orthosis 100 and a reduction of pressure marks at the finger due to the frame 1. The finger orthosis 100 of the third embodiment is, unlike the finger orthosis 100 of FIG. 1, completely closed around the region of the fingertip.

FIG. 4 shows a plan view of a fourth embodiment of a finger orthosis 100 in accordance with the invention. The finger orthosis 100 of the fourth embodiment is a finger orthosis 100 as has already been described with reference to FIG. 1 with the difference that the finger orthosis 100 of FIG. 4 is configured for a thumb 2. The finger orthosis 100 for a thumb 2 likewise has a frame 1 that is closed in a plane in parallel with the upper side 6 and in a peripheral direction 5 in the region of the thumb tip 2a. With the exception of a gap 1a, the frame 1 is largely closed at a metacarpophalangeal member side of the thumb in the region for receiving a distal interphalangeal joint of the thumb 2b. The frame 1 is flexible in the region of the distal interphalangeal joint of the thumb 2b due to the gap 1a and adapts to different thumb circumferences. In the view shown here, a passage opening 1b of the frame 1 can additionally be recognized in the region of the thumb tip 2a, a passage opening 1c of the frame 1 at the upper side 6 in the region of the distal interphalangeal joint of the thumb 2b, and lateral passage openings 1g on a side of the metacarpophalangeal member of the thumb of the frame 1 in the region of the distal interphalangeal joint of the thumb. In the region of the thumb tip 2a, a cable connector holder as a tool 4, as in FIG. 1, is fastened to the frame 1 by means of adhesive bonding or laminating.

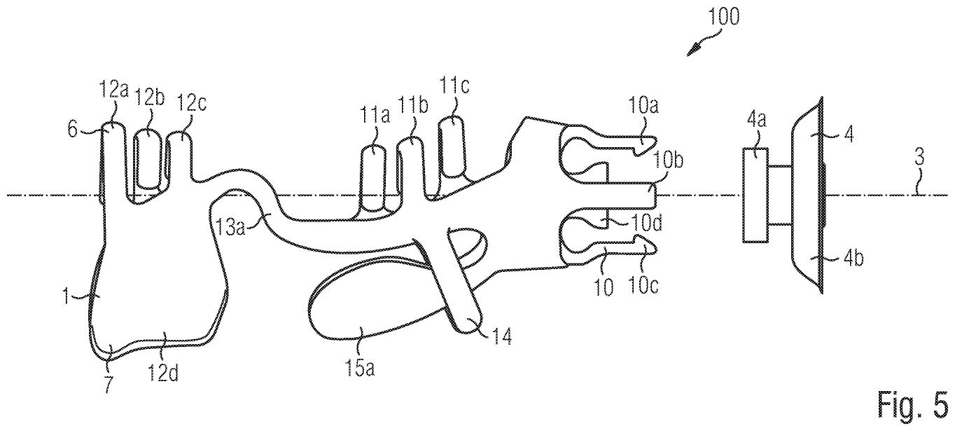

FIG. 5 shows a side view of a finger orthosis 100 having a frame 1 and a tool mount 10 into which a tool 4, likewise shown in FIG. 5, can be plugged. The tool 4 is here latched with the tool mount 10. The tool 4 has a round latch element 4a and a carbon roller 4b by which the workman can carry out a plugging procedure in a facilitated manner. The tool mount 10 is formed in one piece with the frame 1. It can, however, also be manufactured separately and be connected to the frame 1, for example by welding or adhesive bonding. In FIG. 5, the carbon roller 4b and the tool 4 are in turn latchable to the tool mount via latch noses 10a, 10b, 10c, and 10d of the tool mount 10 and a latching element 4a and can thus be fixed in a shape matching manner.

The frame has three brackets 11a, 11b, and 11c at the end facing or close to the fingertip, said three brackets 11a, 11b, and 11c surrounding the finger in a plane perpendicular to the longitudinal direction 3 of the finger and of the finger orthosis 100. The brackets are connected to the rest of the frame only at one side alternately at both sides and are thereby flexible. They can thus adapt to the circumference of a finger and can also flexibly follow the surrounded finger member on movements of the finger in the joint. They together form a cuff that should engage around the finger and should fix the orthosis 100 at the finger.

In a similar manner, three brackets 12a, 12b, and 12c are part of the frame at the end of the orthosis close to the finger joint. A further cuff 12d that is provided at the lower side 7 of the orthosis 100 to engage around the finger forms, together with the brackets 12a, 12b, and 12c, a cuff engaging around the entire finger in a plane perpendicular to the longitudinal axis 3.

Two connection elements 13a and 13b extend laterally of the finger or of the orthosis 100 between the regions having the brackets 11a, 11b, 11c and 12a, 12b, 12c and connect these regions to one another. These connection elements 13a and 13b can be designed such that they limit the angle at which the finger joint can be bent. Alternatively, it can also only be signaled to the workman by an increased resistance of the sections 13a and 13b that he should not bend the finger further. Unergonomic positions of the finger joint are prevented or signaled in this manner.

A bracket 14 that is intended to engage around between both sides and that is designed with greater material thickness or more stable material is arranged at the lower side 7 of the orthosis 100. It serves as a support for adjacent fingers that thus support the finger surrounded by the orthosis 100 and the orthosis. This makes it possible to lead off or distribute the forces acting on the orthosis over further fingers or over the whole hand.

Two sections 15a and 15b (not recognizable in FIG. 5) are provided within this bracket and extend freely projecting from the tool mount 10 or from the tip of the orthosis 100 below the finger or on the lower side 7 of the orthosis 100 in the direction of the finger joint or of the cuff 12d. These sections extend up to the region of the orthosis 100 that is intended to surround the finger joint. They serve to improve the feeling of the workman when gripping. They can also serve to limit the angle at which the finger joint can be bent. Alternatively, it can also only be signaled to the workman by an increased resistance of the sections 15a and 15b that he should not bend the finger further. Unergonomic positions of the finger joint are prevented or signaled in this manner.

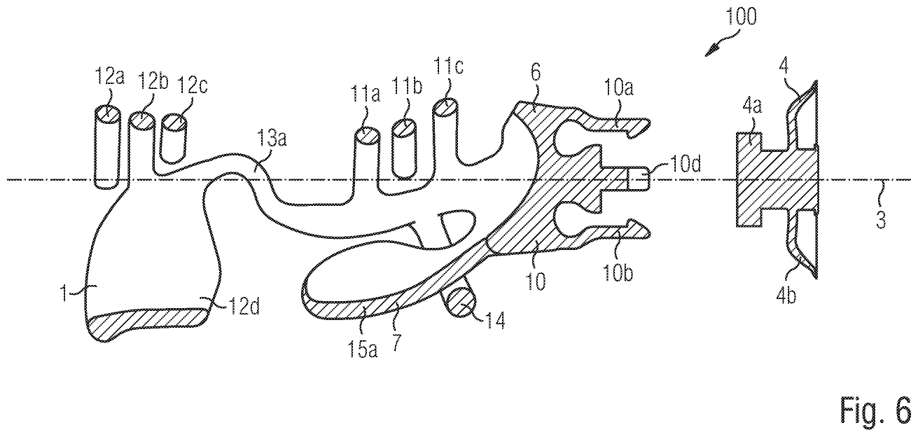

FIG. 6 shows the finger orthosis 100 in FIG. 5 in a sectional view. The tool mount 10 is now not formed in one piece with the frame 1, but is rather manufactured separately and connected to the frame 1, for example by welding or adhesive bonding. The carbon roller is in turn latchable to the tool mount via latch noses 10a, 10b, 10c, and 10d at the tool mount 10 and a latching element 4a and can thus be fixed in a shape matching manner.

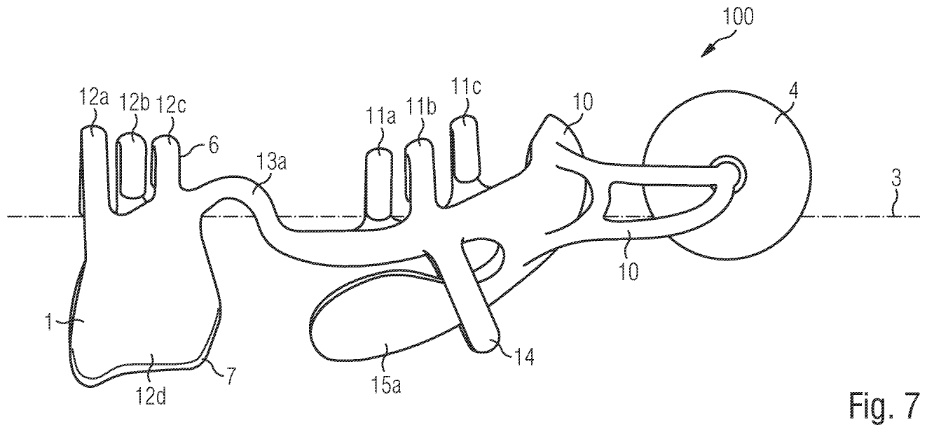

FIG. 7 shows a side view of a further finger orthosis 100 having a frame 1. At the tip of the finger orthosis 100, a holding apparatus 10 having two webs that are connected to one another and at whose end a rotatable wheel 4 is arranged as a tool is located. The wheel 4 can be a cutting blade, for example. An object can then be cut by the finger orthosis 100, with a sufficient safety distance between the hand of the workman and the tool 4 being ensured by the finger orthosis 100 and the holding apparatus 10.

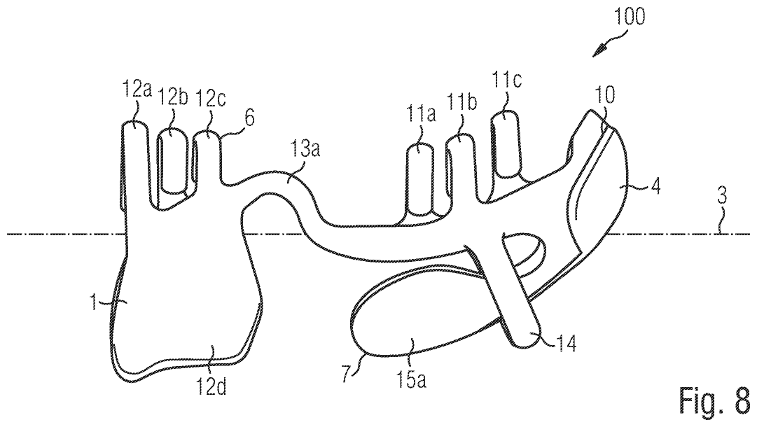

FIG. 8 shows a side view of a further finger orthosis 100 having a frame 1 and a tool 4. The tool 4 is here a pad of an elastic material by which pressure can be exerted on a workpiece. The finger of the workman is protected by the elastic material of the tool 4 here. The embodiment of the frame 1 has a large opening at the rear side for the finger or for the knuckle. The orthosis 1 can thereby be easily bent together with the finger that receives the orthosis.

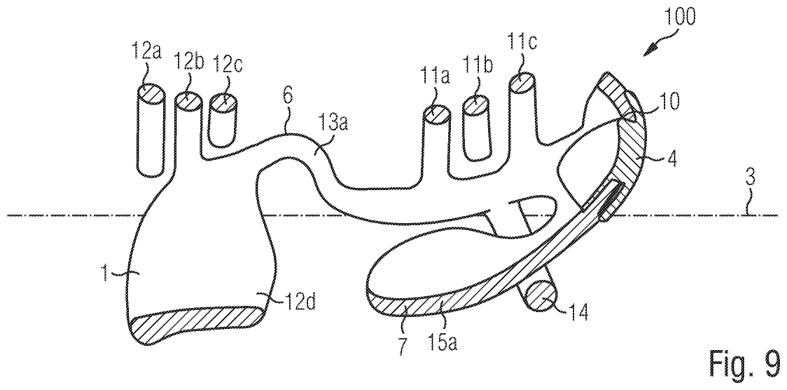

FIG. 9 shows a sectional side view of the finger orthosis 100 of FIG. 8. The elastic pad 4 is introduced into a tool mount 10 in the form of an opening within the frame 1 and is there fixed in a shape matching manner to the tool mount 10 and the frame 1 by an undercut behind the circumferential margin of the opening.

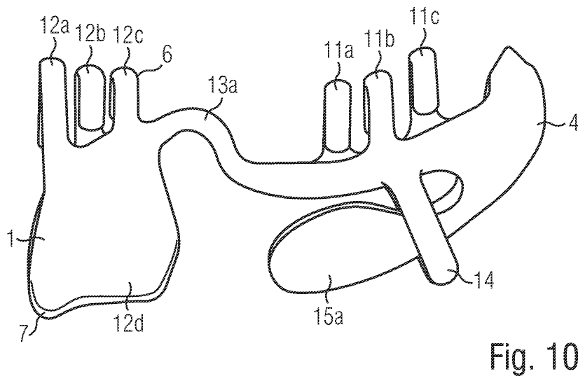

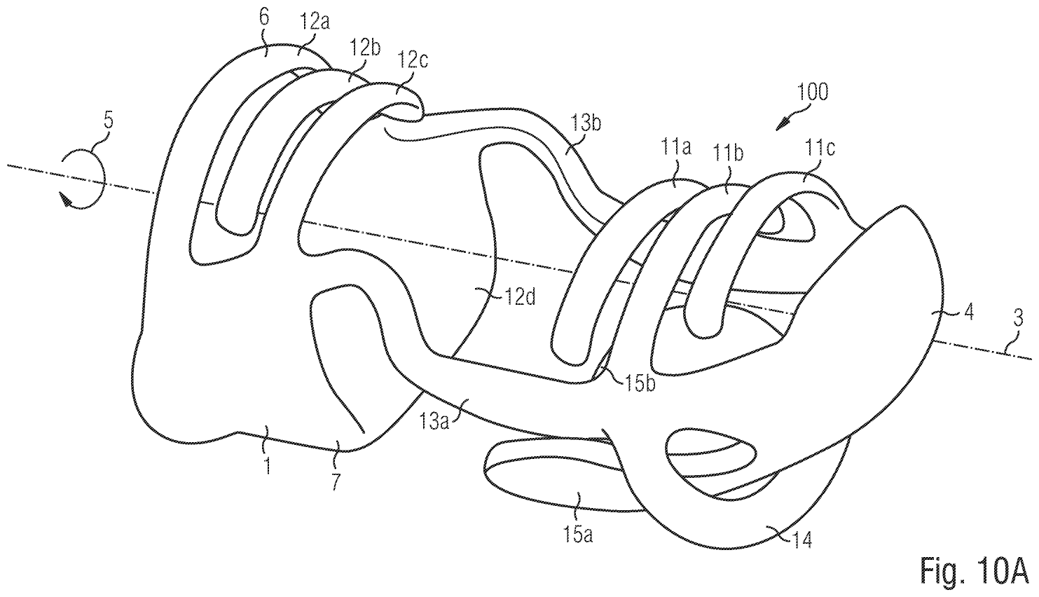

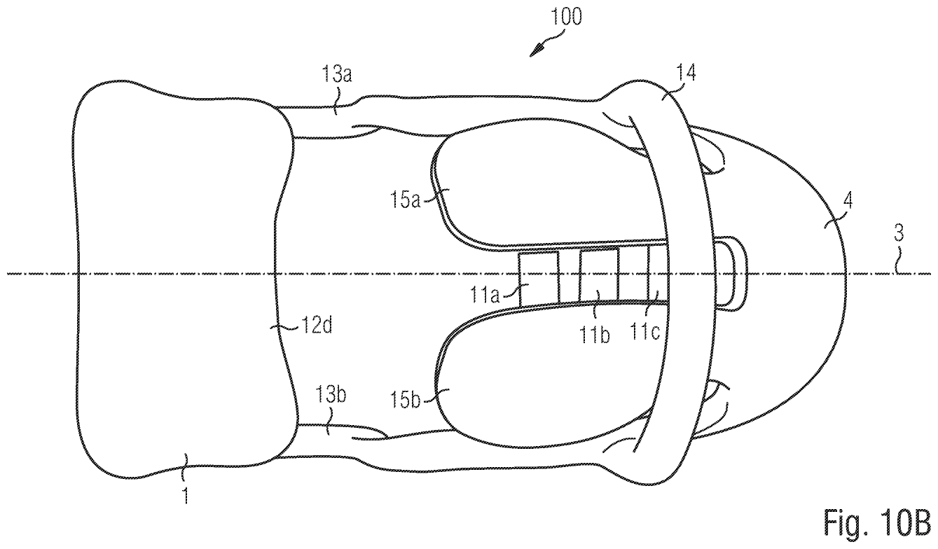

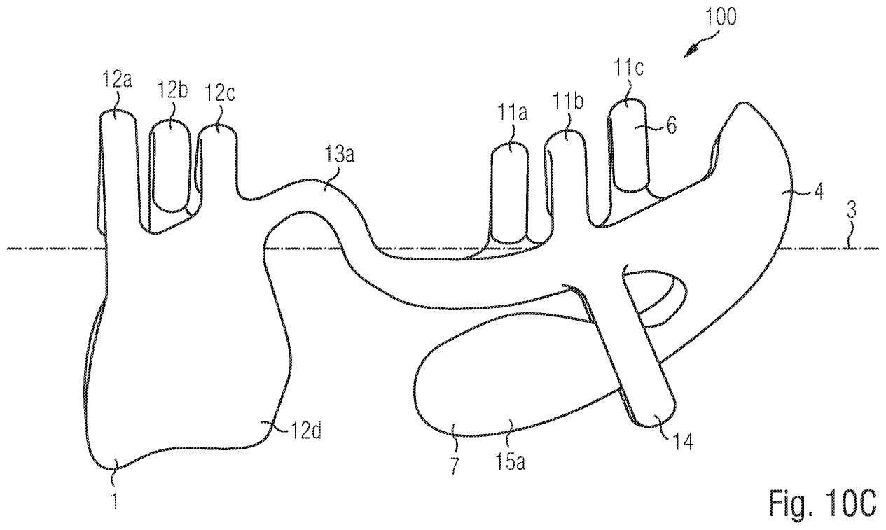

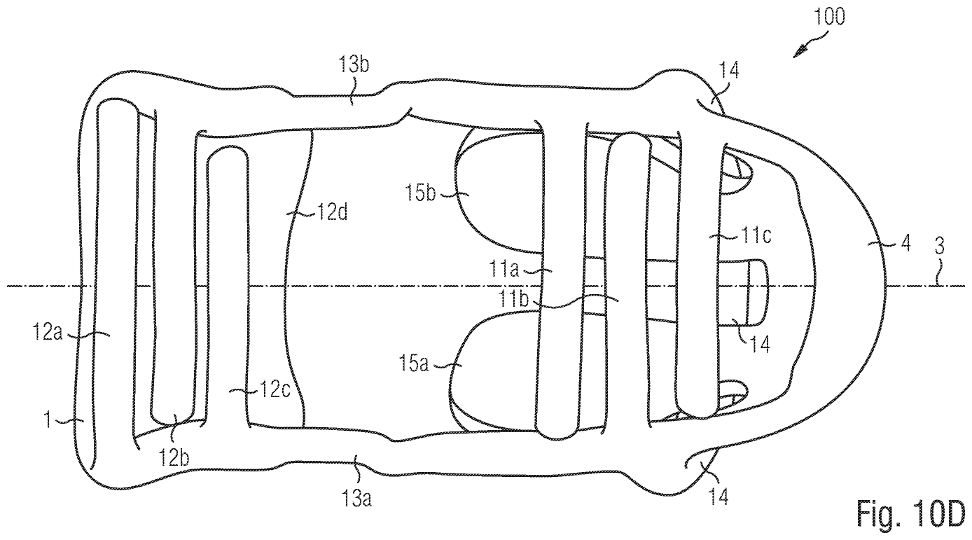

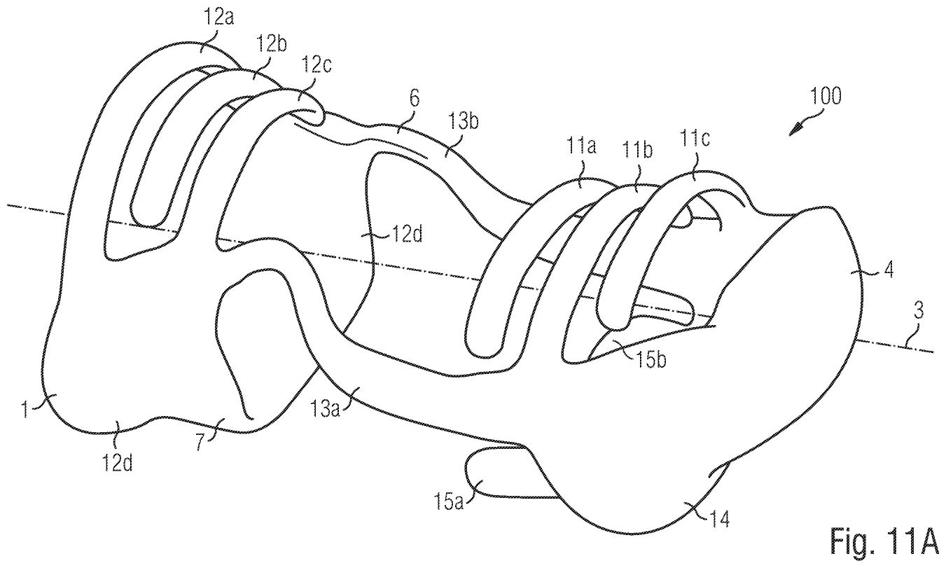

FIG. 10 shows a side view of a further finger orthosis 100 having a frame 1. In a similar manner to the finger orthosis 100 in FIG. 7, a tool 4 in the form of a pad is arranged at the end of the frame 1 corresponding to the fingertip. This pad 4 is formed in one piece with the frame 1 and from the same material in FIG. 10. The whole frame 1 can consequently be formed from the material of the pad 4. The tool characteristic of the section 4 of the frame 1 therefore results from the use of this section for machining, treating, or acting on a component. Such a finger orthosis 100 is suitable, when selecting an appropriate material, for example, to exert great pressure on a component, with the stress of the finger of the workman being limited. It is also suitable to operate a capacitive screen. As a further possibility, for an inductive screen, corresponding electronic components that enable the operation of a capacitive screen are integrated in the region of the tool 4 of the frame 1 as a further possibility.

FIGS. 10A to 10D show a lateral plan view (FIG. 10A), a lower view (FIG. 10B), a lateral view (FIG. 10C), and a plan view from above (FIG. 10D) of the finger orthosis 100.

It can be recognized in FIG. 10B that two regions 15a and 15b are provided that both extend, viewed in the plan view of FIG. 10B, next to one another at a respective side of the longitudinal axis 3 in the direction of the longitudinal axis 3.

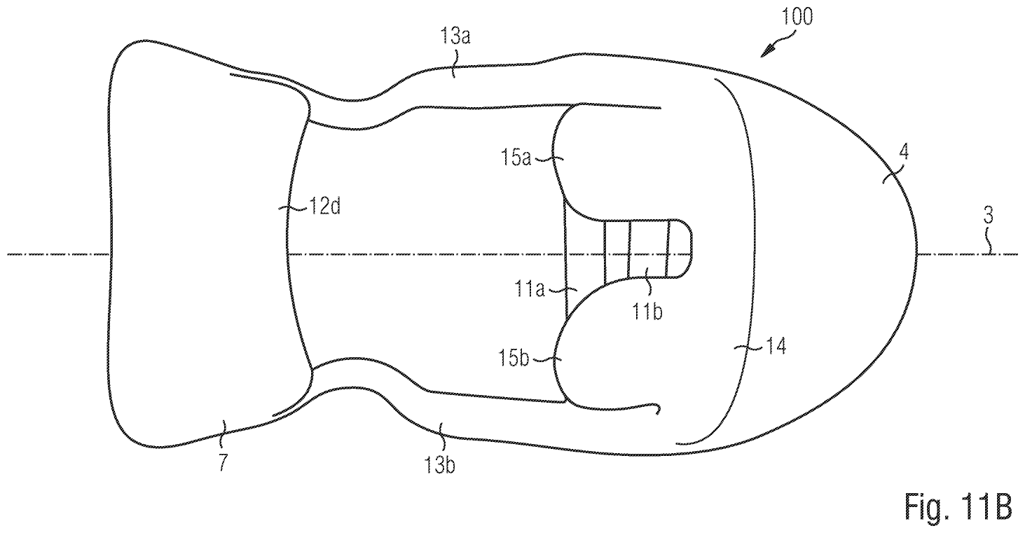

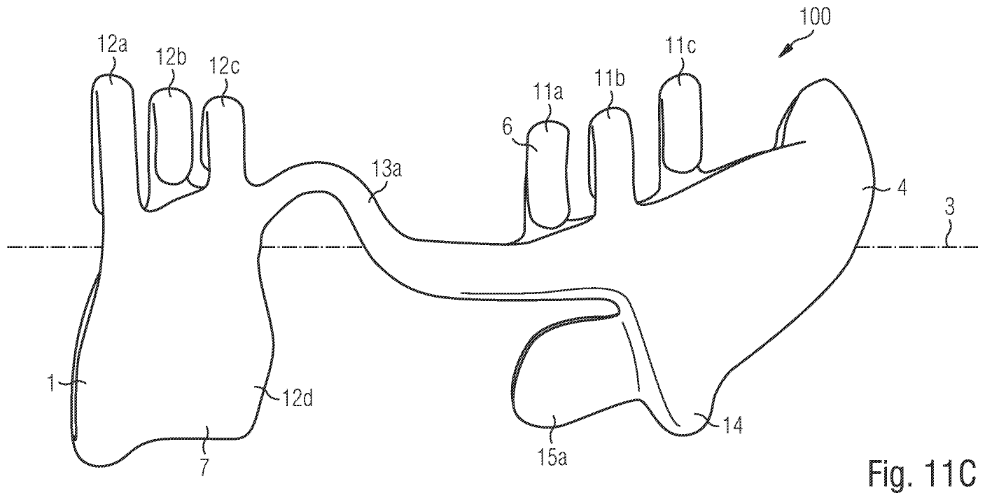

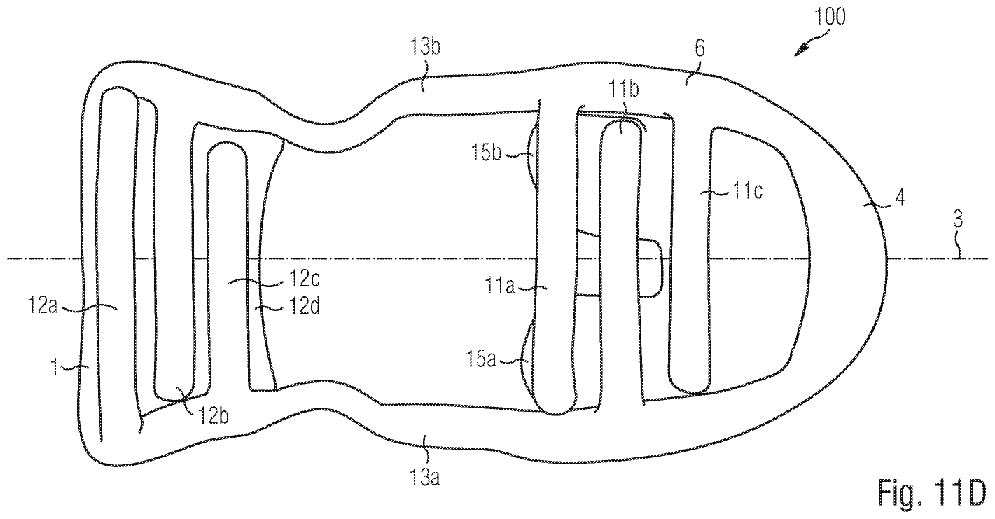

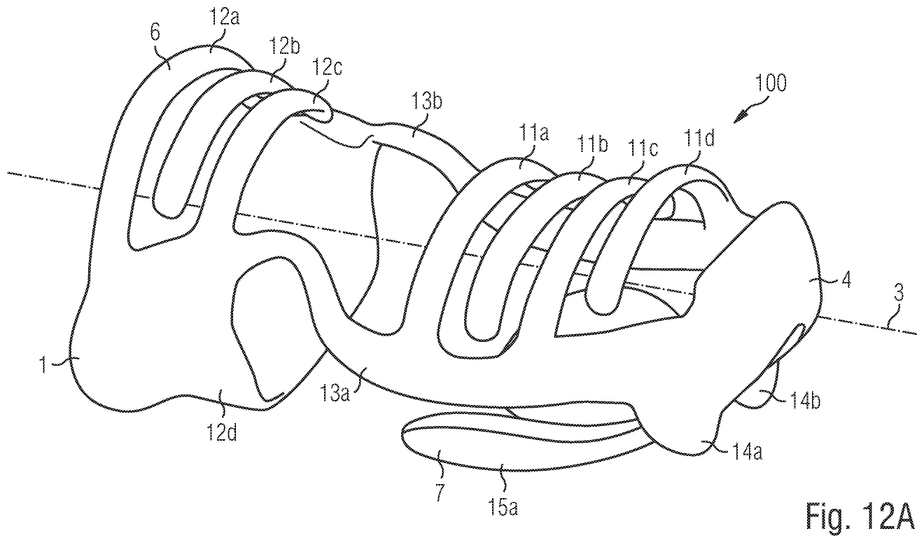

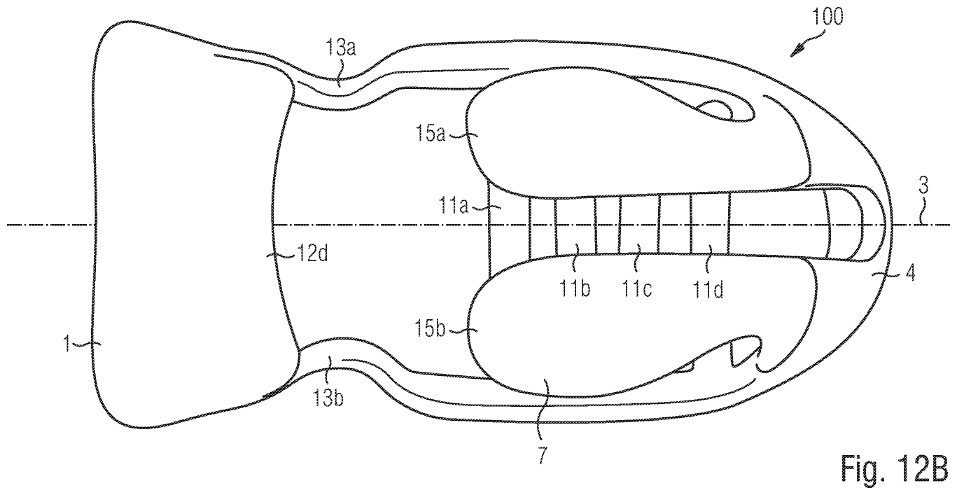

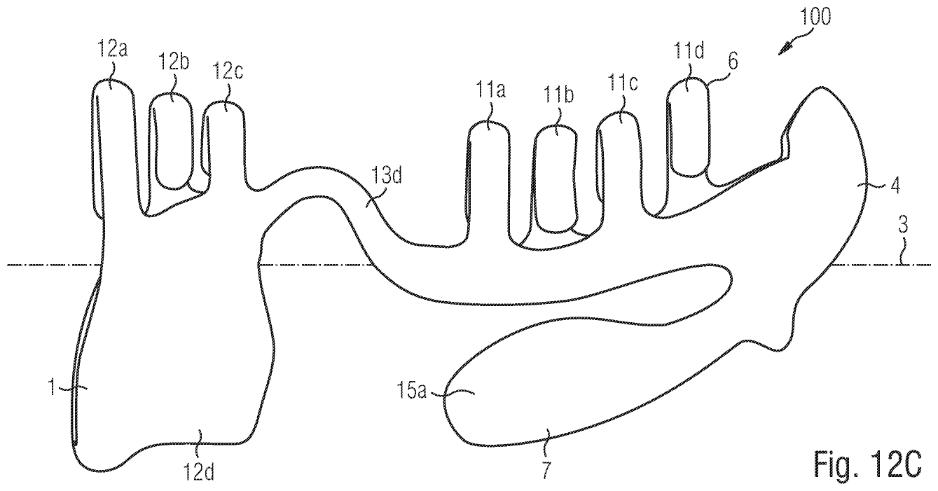

FIGS. 11A to 11D show a further finger orthosis 100 similar to that in FIGS. 10 to 10D in an oblique plan view (FIG. 11A), a lower view (FIG. 11B), a lateral plan view (FIG. 11C) and a plan view from above (FIG. 11D). Differing from this, in FIGS. 11A to 11D, the bracket 14 is integrated in the regions 15a and 15b and is characterized by a greater thickness than the other sections of the regions 15a and 15b. In other words, the bracket is no longer arranged in a self-supporting manner between the two sides of the orthosis 100, but rather between the regions 15a, 15b and the tip of the orthosis 100.

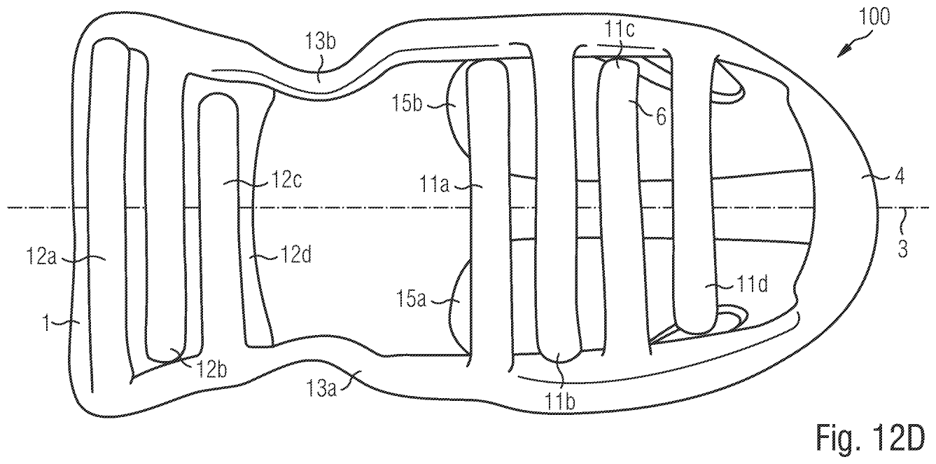

FIGS. 12A to 12D show a further finger orthosis 100 similar to that in FIGS. 10 to 10D in an oblique plan view (FIG. 12A), a lower view (FIG. 12B), a lateral plan view (FIG. 12C) and a plan view from above (FIG. 12D). Differing from this, however, four freely projecting brackets 11a to 11d are now provided instead of three freely projecting brackets 11a to 11c. Furthermore, in addition to the regions 15a and 15b, elements 16a and 16b are provided that are each arranged as support elements for adjacent fingers instead of the bracket 14 in FIGS. 11A to 11D between one of the regions 15a, 15b and the tool 4.

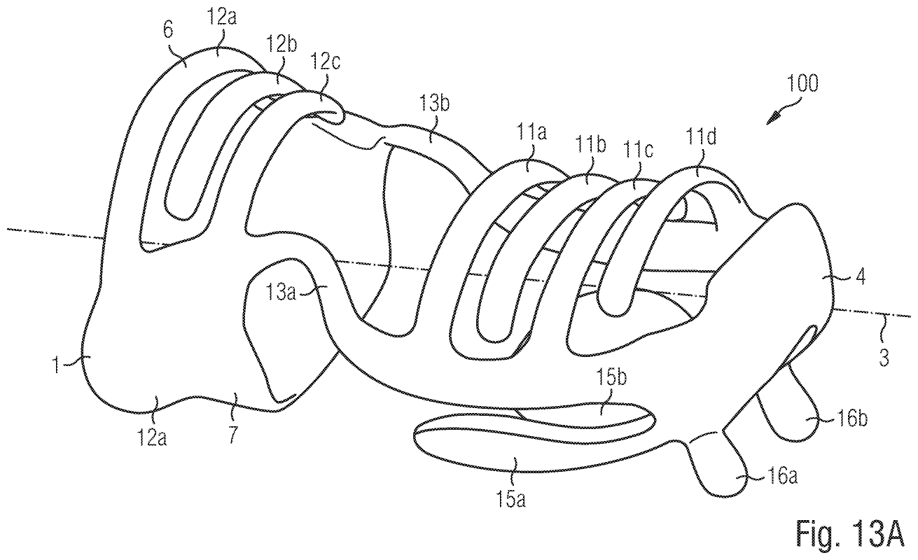

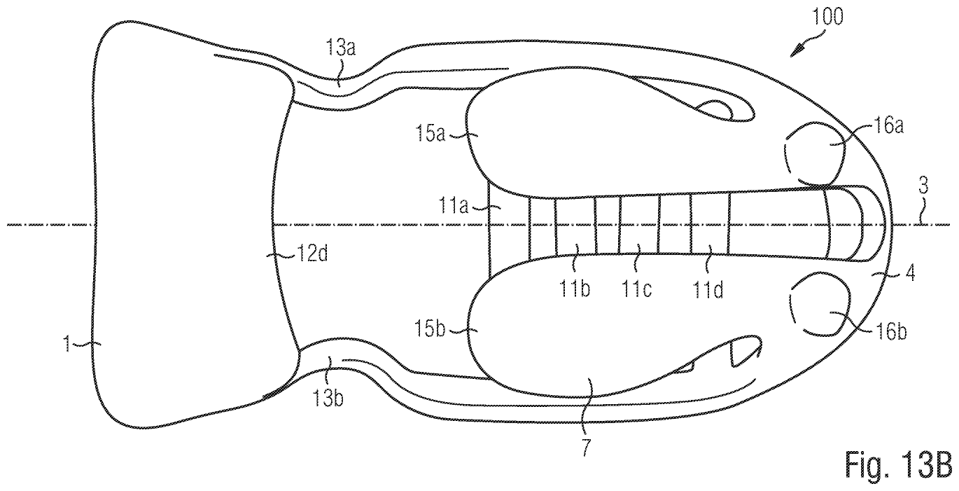

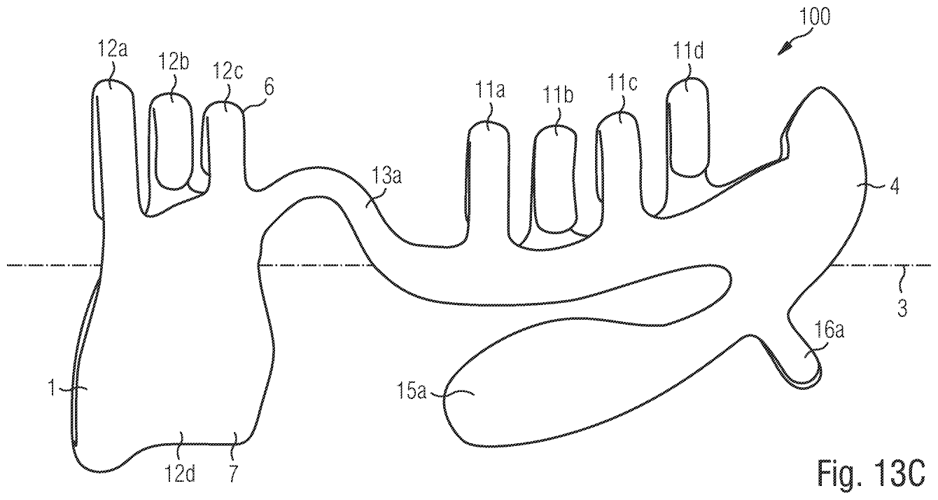

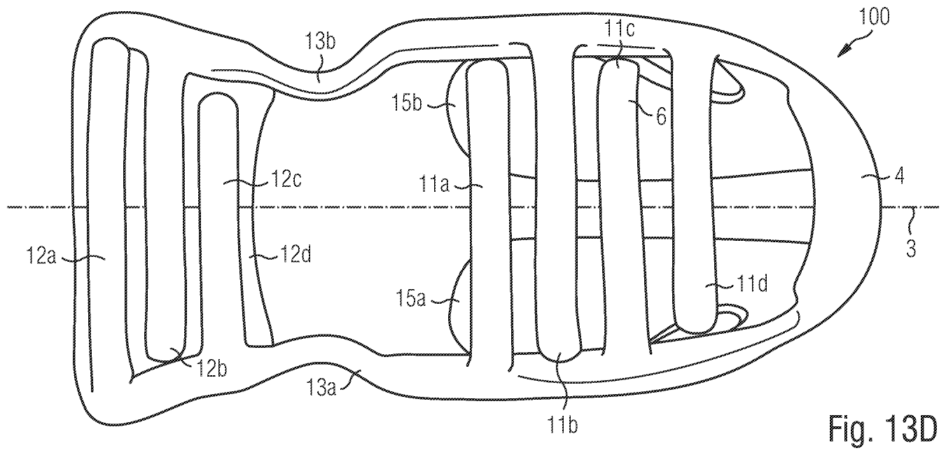

FIGS. 13A to 13D show a further finger orthosis 100 similar to that in FIGS. 12A to 12D in an oblique plan view (FIG. 13A), a lower view (FIG. 13B), a lateral plan view (FIG. 13C) and a plan view from above (FIG. 13D). Differing from this, however, the support elements 16a, 16b project further from the adjacent surface of the finger orthosis 100.

1. A finger orthosis for exerting a force on a component by means of a finger comprising:
a frame for receiving a finger, wherein the frame,
at least partially circularly surrounds a fingertip and a side of a distal interphalangeal joint remote from the fingertip in each case substantially perpendicular to a longitudinal direction of the finger; and
at least partially circularly surrounds the finger from the fingertip to the side of the distal interphalangeal joint remote from the fingertip substantially in a plane of the longitudinal direction, and
a tool connected to the frame for transferring the force onto the component.

2. A finger orthosis in accordance with claim 1, wherein the frame has a support element for supporting the finger orthosis on at least one further finger.

3. A finger orthosis in accordance with claim 1, wherein the support element has at least one bracket that engages around the finger on its ventral side and is connected to the frame on at least one side of the finger.

4. A finger orthosis for receiving a finger comprising:
a support element for supporting the finger orthosis on at least one further finger.

5. A finger orthosis in accordance with claim 4, further comprising:
a frame for receiving a finger, wherein the frame,
at least partially circularly surrounds a fingertip and a side of a distal interphalangeal joint remote from the fingertip in each case substantially perpendicular to a longitudinal direction of the finger; and
at least partially circularly surrounds the finger from the fingertip to the side of the distal interphalangeal joint remote from the fingertip substantially in a plane of the longitudinal direction.

6. A finger orthosis in accordance with claim 4, wherein the support element has at least one bracket that engages around the finger on its ventral side and is connected to the frame on at least one side of the finger.

7. A finger orthosis in accordance with claim 4, wherein the frame has a tool connected to the frame for transferring a force onto a component.

8. A finger orthosis in accordance with claim 4, wherein the support element has a thickness of at least 0.75 mm, advantageously at least 3 mm;
comprises one or more of the following materials: plastic, metal, fiber composite, carbon, and silicone.

9. A finger orthosis in accordance with claim 4, wherein the frame has a section that extends on the front side of the finger over the finger joint such that it limits the flexion of the finger joint.

10. A finger orthosis in accordance with claim 9, wherein the section is connected to the frame at the fingertip and extends as a web over the finger joint.

11. A finger orthosis in accordance with claim 4, wherein the frame is produced in one piece.

12. A finger orthosis in accordance with claim 4, wherein the frame has a tool connected to the frame for transferring a force to a component, with the frame and the tool being at least one of flexibly, rigidly, or directly connectable or connected to one another or produced in one piece with one another.

13. A finger orthosis in accordance with claim 4, wherein the tool is connected to the frame via a tool mount produced in one piece with the frame.

14. A finger orthosis in accordance with claim 4, wherein the frame is at least one of formed over the full area over the surface spanned by it or has one or more passage openings.

15. A finger orthosis in accordance with claim 4, wherein within the frame a wall is arranged that is firmly connected to the frame and that extends over a surface of the finger orthosis spanned by the frame.

16. A finger orthosis in accordance with claim 4, wherein the wall is at least one of closed over the full area over the surface spanned by the frame or has one or more passage openings, with the passage openings in particular corresponding to the passage openings of the frame.

17. A method of manufacturing a finger orthosis in accordance with claim 4, comprising:
producing the frame using at least one of a 3D printing process, an injection molding process, and/or a laminate process; and
fastening the tool to the frame or at least one of producing the tool together or in one piece with the frame.

18. A method in accordance with claim 4, wherein, for the fastening of the tool, a tool mount is at least one of adhesively bonded, plugged, or screwed to the frame or is produced in one part with the frame and the tool is fastened to the tool mount or is produced in one part with the tool mount.

19. A method in accordance with claim 17, wherein the tool is directly adhesively bonded, plugged, or screwed to the frame, or flexibly connected to the frame or is produced in one part with the frame.
